(19) United States
(12) Patent Application Publication
(10) Pub. No.: US 2012/0071571 A1
(43) Pub. Date: Mar. 22, 2012

(54) COBALT CATALYSTS

(75) Inventors: Hamera Abbas, Rochdale (GB); Sharon Bale, Stockton on Tees (GB); Gordon James Kelly, Darlington (GB); John West, Darlington (GB)

(73) Assignee: JOHNSON MATTHEY PLC, London (GB)

(21) Appl. No.: 13/126,306

(22) PCT Filed: Oct. 1, 2009
(86) PCT No.: PCT/GB2009/051288

§ 371 Date: Oct. 24, 2011

(30) Foreign Application Priority Data
Oct. 30, 2008 (GB) 0819847.5

(51) Int. Cl. B01J 37/16; B01J 31/28; C07C 1/04; B01J 21/04; B01J 37/18; B82Y 40/00; B82Y 30/00

(52) U.S. Cl. 518/700; 502/332; 502/327; 502/330; 502/328; 502/329; 502/150; 518/715; 977/773; 977/895

(57) ABSTRACT

A catalyst suitable for the Fischer-Tropsch synthesis of hydrocarbons is described comprising cobalt nanocrystallites containing a precious metal promoter, dispersed over the surface of a porous transition alumina powder wherein the cobalt content of the catalyst is ≧25% by weight, the precious metal promoter metal promoter content of the catalyst is in the range 0.05 to 0.25% by weight, and the cobalt crystallites have a average size, as determined by hydrogen chemisorption, of ≧15 nm. A method for making the catalyst is also described.

### CROSS-REFERENCE TO RELATED APPLICATIONS

This application is the U.S. National Phase application of PCT International Application No. PCT/GB2009/051288, filed Oct. 1, 2009, and claims priority of British Patent Application No. 0819847.5, filed Oct. 30, 2008, the disclosures of both of which are incorporated herein by reference in their entirety for all purposes.

### FIELD OF THE INVENTION

This invention relates to cobalt catalysts and in particular precious metal promoted cobalt catalysts supported on a transition alumina, suitable for use in the Fischer-Tropsch synthesis of hydrocarbons.

### BACKGROUND OF THE INVENTION

Precious metal-promoted cobalt Fischer-Tropsch catalysts supported on titania alumina or silica are known. U.S. Pat. No. 4,088,671 discloses a hydrocarbon synthesis process using Ru-promoted Co catalysts on various supports. U.S. Pat. No. 4,493,905 discloses fluidized bed catalysts suitable for the Fischer-Tropsch reaction prepared by contacting finely divided alumina with an aqueous impregnation solution of a cobalt salt, drying the impregnated support and thereafter contacting the support with a nonaqueous, organic impregnation solution of salts of ruthenium and a Group IIIB or IVB metal. U.S. Pat. No. 4,822,824 discloses Ru-promoted Co catalysts on titania. U.S. Pat. No. 5,302,622 discloses a process for the synthesis of hydrocarbons, using a catalyst comprising cobalt, copper and ruthenium on silica or alumina.

A recurring problem with these catalysts is the rapid initial deactivation observed in use and the associated loss in yield. Moreover, the deactivation is not readily predictable.

### SUMMARY OF THE INVENTION

Heretofore, the focus of researchers has generally been on maximising the activity for the minimum amount of cobalt and this has led to catalysts with increased precious metal promoter levels and smaller cobalt crystallite sizes. We have found surprisingly that by increasing the cobalt content of the catalyst, limiting the amount of precious metal promoter, and subjecting the catalyst precursor to high-temperature reduction step, a larger cobalt crystallite material may be prepared which has both the required activity and selectivity and can give improved stability compared to known catalysts.

Accordingly the invention provides a catalyst suitable for the Fischer-Tropsch synthesis of hydrocarbons comprising cobalt nanocrystallites containing a precious metal promoter, dispersed over the surface of a porous transition alumina powder wherein the cobalt content of the catalyst is ≧25% by weight, the precious metal promoter content of the catalyst is in the range 0.05 to 0.25% by weight, and the cobalt crystallites have a average size, as determined by hydrogen chemisorption, of ≧15 nm.

The invention further provides a process for preparing a catalyst comprising the steps of:

(a) forming a catalyst precursor by;
(i) impregnating a transition alumina with a cobalt compound and precious metal promoter compound,
(ii) drying the impregnated alumina,
   (b) calcining the dried catalyst precursor, and
   (c) reducing the calcined precursor,
   wherein steps (i) and (ii) are performed until the cobalt content of the catalyst is ≧25% by weight and the precious metal promoter content of the catalyst is in the range 0.05 to 0.25% by weight, the calcination is performed at a temperature in the range 250-650° C. and reduction is performed at a temperature in the range 450-650° C., such that cobalt crystallites in the catalyst have a average size, as determined by hydrogen chemisorption, of ≧15 nm.

The invention further provides a process for the Fischer-Tropsch synthesis of hydrocarbons comprising the step of passing a gas mixture comprising hydrogen and carbon monoxide over the catalyst.

### BRIEF DESCRIPTION OF THE FIGURES

The invention is further illustrated by reference to the drawings in which:

FIG. 1 shows a Temperature Programmed Reduction (TPR) plot profile of catalyst samples P2 and C2; and

FIG. 2 shows a Temperature Programmed Reduction (TPR) plot profile of catalyst samples P3 and C3.

### DETAILED DESCRIPTION OF THE INVENTION

By “cobalt crystallites” we mean cobalt metal crystallites and not cobalt oxide present in the catalyst precursor.

Deliberately setting out to create large cobalt crystallites appears counter-intuitive insofar as previous work has indicated that activity in the Fischer-Tropsch reaction can be proportional to cobalt surface area, which drops as the crystallite size increases. Indeed, a recent publication has suggested that the optimum average cobalt crystallite size for selectivity is in the region of 7-8 nm. (see J. Catal. 259, (2), 2008, pages 161-164).

Moreover, the beneficial high temperature reduction is surprising in view of previous published work that indicates that reduction temperature has only a negligible effect on catalyst activity. For example, in J. Catalysis, 166, 1997, pages 8-15, the authors examined a 0.5% wt Ru-promoted 20% wt Co/alumina catalyst with calcination in air at 200-400° C. and reduction in hydrogen at 300-400° C. showed that while calcination temperature had a pronounced negative effect on the overall activity, the reduction temperature had only a negligible effect.

In the present invention, the catalyst, i.e. the reduced catalyst, has a cobalt content ≧25% by weight, preferably in the range 25-45% by weight to keep the number of impregnations down during manufacture.

The promoter metal may be selected from one or more of Pt, Pd, Re, Ru, Ir or Au, however, Ru is particularly preferred. The optimal amount of Ru has been found to be in the range 0.05 to 0.15% wt, which is considerably lower than in many of the catalysts previously tested. Lower Ru levels clearly have beneficial handling and cost implications.

If desired, the catalyst may in addition to cobalt and precious metal promoter, further comprise one or more suitable additives useful in Fischer-Tropsch catalysis. For example, the catalyst may comprise one or more additives that alter the physical properties and/or promoters that effect the reducibility or activity or selectivity of the catalysts. Suitable additives are selected from compounds of metals selected from molybdenum (Mo), iron (Fe), manganese (Mn), titanium (Ti), zirconium (Zr), lanthanum (La), cerium (Ce), chromium (Cr), magnesium (Mg) or zinc (Zn). Typical amounts of the additives are 0.1-10% metal by weight on calcined catalyst precursor. If desired, the compounds of the additional additives may be added in suitable amounts to the cobalt and/or precious metal promoter solution. Alternatively, they may be combined with the catalyst precursor before or after drying or calcination. However, in the present invention, it has been possible to provide suitably active and stable catalysts without the inclusion of such additives.

The amount of cobalt, precious metal promoter and additive (if included) in the catalyst or catalyst precursor may be readily determined using known methods, e.g. ICP-Atomic emission spectroscopy.

The transition alumina may be of the gamma-alumina group, for example a eta-alumina or chi-alumina. These materials may be formed by calcination of aluminium hydroxides at 400 to 750° C. and generally have a BET surface area in the range 150 to 400 m²/g. Alternatively, the transition alumina may be of the delta-alumina group, which includes the high temperature forms such as delta- and theta-aluminas that may be formed by heating a gamma group alumina to a temperature above about 800° C. The delta-group aluminas generally have a BET surface area in the range 50 to 150 m²/g. In the present invention, the transition alumina preferably comprises gamma alumina and/or a delta alumina with a BET surface area in the range 120-160 m²/g. Where the catalyst precursor is prepared using a gamma alumina, it is possible by the calcination and reduction procedure to convert at least a portion of this to delta alumina. Thus the catalyst precursor may be prepared with a gamma alumina yet the catalyst comprise precious-metal promoted cobalt crystallites dispersed over a gamma alumina, a delta alumina or a mixed phase material comprising delta and gamma aluminas. The alumina should be of suitable purity for use as a catalyst support. In particular the level of alkali metal, notably sodium, in the alumina is desirably <50 ppm, more preferably <10 ppm.

A suitable alumina for the catalyst support generally has a volume-median diameter D[v,0.5] in the range 1 to 200 μm. In certain applications such as for catalysts intended for use in slurry reactions, it is advantageous to use very fine particles which have a volume-median diameter D[v,0.5], in the range from 1 to 30 μm, e.g. 5 to 25 μm. For other applications e.g. as a catalyst for reactions carried out in a fluidised bed, it may be desirable to use larger particle sizes, preferably in the range 50 to 150 μm. The term volume-median diameter D[v,0.5], sometimes given as D₅₀ or D_{0.5}, is defined by Dr Alan Rawle in the paper “Basic Principles of Particle Size Analysis” available from Malvern Instruments Ltd, Malvern, UK (see www.malvern.co.uk), and is calculated from the particle size analysis which may conveniently be effected by laser diffraction for example using a Malvern Mastersizer.

The pore volume of the alumina support is preferably relatively high in order to take the high cobalt loadings. The pore volume of the alumina is preferably above 0.30 cm³/g, more preferably in the range 0.35 to 0.65 cm³/g, and may be determined by nitrogen physisorption using known techniques. It is preferred that the alumina support has a relatively large average pore diameter as the use of such supports may give catalysts of particularly good selectivity. Preferred supports have an average pore diameter (APD) of at least 10 nm, particularly in the range 12 to 20 nm. [By the term average pore diameter we mean 4 times the pore volume as measured from the adsorption branch of the nitrogen physisorption isotherm at 0.99 relative pressure divided by the BET surface area].

During the production of the compositions of the invention, cobalt compounds are deposited in the pores of the support, and so the average pore diameter of the catalyst will be less than that of the alumina employed, and decreases as the proportion of cobalt increases. It is preferred that the catalysts have an average pore diameter of at least 8 nm and particularly in the range 10 to 20 nm.

When the support is transition alumina, it has been found that the bulk of the cobalt can be deposited as cobalt compounds within the pores of the transition alumina and none or only a small proportion of the cobalt is deposited as a coating round the alumina particles. As a result, the particle size of the catalysts of the invention is essentially the same as the particle size of the transition alumina support, and so the catalysts of the invention generally may have a volume-median diameter D[v,0.5] in the range 1 to 200 μm, in one embodiment preferably less than 30 μm and particularly less than 25 μm, and in a second embodiment preferably in the range 50 to 150 μm.

In a particularly preferred embodiment, the catalyst has a volume-median diameter D[v,0.5] in the range 15 to 25 μm. To achieve this the transition alumina powder also preferably has a volume-median diameter D[v,0.5] in the range 15 to 25 μm.

Moreover, since the cobalt compounds are primarily deposited within the pores of the transition alumina support, the pore volume of the catalysts in accordance with the invention will be less than that of the support employed, and will tend to decrease as the cobalt loading increases. Catalysts having a total cobalt content in the range 25 to 45% by weight preferably have a pore volume of at least 0.2 ml/g.

The compositions of the invention, when in the reduced state comprise cobalt crystallites that have an average size of ≧15 nanometres (nm), i.e. the cobalt crystallites have an average diameter or width ≧15 nm. Preferably the average crystallite size is ≧17.5 nm, more preferably ≧20 nm. The maximum average crystallite size will depend upon the desired balance of properties but may be ≦40 nm, preferably ≦30 nm. Average cobalt crystallite size may be determined from the cobalt surface area measurement, which may be suitably determined by hydrogen chemisorption. The basis for determining the cobalt crystallites size in the present invention is set out as follows;

### (a) Relationship Between Average Particle Size and Dispersion.

The Co crystallite size for a given Co/Al₂O₃ catalyst can be determined by re-arranging some fundamental equations. The mean surface average diameter, <d>, is related to the dispersion by the equation:

$$d = \frac{f \times A \times 100}{ρ \times {S}_{A} \times N} \times \frac{S}{V} \qquad {( 1 )}^{\neq }$$

Where:

d=dispersion of Co atoms (% atoms). Dispersion is a percentage of the cobalt metal atoms on the surface of the crystallites/total Co metal atoms present.
f=fraction of the surface area effectively exposed
A=atomic weight of the atom (58.93 for Co)
ρ=the specific mass density (ρ_{Co}=8.9 g/cm³=8.90×10⁻²¹ g/nm³)^{†}
S_{A}=surface area occupied by one Co atom at the surface (assumed as 0.0662 nm² for Co)^{§}
N=Avogadro's number (6.022×10²³)
S=total surface area of the active phase
V=total volume of the active phase

Given that the volume of a sphere is V=4/3 πr³ and that the surface area is S=4 πr², then assuming identical spheres of diameter <d>, it can be shown that:

$$\frac{S}{V} = \frac{6}{〈 d 〉} \qquad ( 2 )$$

Where <d>=mean surface average diameter.

Assuming that f=1, then equations (1) and (2) can be rearranged to give:

$$〈 d 〉 = \frac{A \times 6 \times 100}{ρ \times {S}_{A} \times N \times d} \\ 〈 d 〉 = \frac{58.93 \times 6 \times 100}{8.9 \times {10}^{- 21} \times 0.0662 \times 6.022 \times {10}^{23} \times d} \qquad ( 3 )$$

Equation (3) can be simplified to:

$$〈 d 〉 = \frac{99.65}{d} ( nm ) \qquad ( 4 )$$

### (b) Relationship Between Co Surface Area and Average Particle Size.

$$\begin{matrix}{S}_{Co} = surface area per gram Co \\ = surface area of 1 Co atom \times d \times \\ No of Co atoms present per gram Co \\ = {S}_{A} \times d \times No of Co atoms present per gram Co\end{matrix}$$

Where:

S_{A}=average size of one atom (for Co=0.0662 nm²=6.62×10⁻²⁰ m²)
d=dispersion of Co (% atoms)

Therefore to calculate S_{Co}:

$${S}_{Co} = 6.62 \times {10}^{- 20} {m}^{2} \times \frac{d}{100} \times \frac{1}{58.93} \times 6.022 \times {10}^{23} \qquad ( 5 )$$

Which can be simplified to:

S_{Co}=6.76d (6)

Therefore:

S_{Co}/6.76=d (7)

Equation (7) can be substituted into equation (4) to give:

$$〈 d 〉 = \frac{99.65}{d } = \frac{99.65}{{S}_{Co} / 6.76} = \frac{673.63}{{S}_{Co}} ( nm ) \qquad ( 8 )$$

S_{Co} can also be calculated in the following way from S_{cat} (Surface area per g catalyst):

$${S}_{Co} = \frac{100}{L} \times {S}_{cat} ( {m}^{2} {g}_{Co}^{- 1} ) \qquad ( 9 )$$

Where:

S_{cat}=Co Surface area per gram of catalyst (m²g_{cat}⁻¹)
L=reduced metal loading (% by weight)

Equations (8) and (9) can be rearranged to give the following relationship between <d> and S_{cat}

$$〈 d 〉 = \frac{L \times 6.74}{{S}_{cat}} \qquad ( 10 )$$

Where the degree of reduction of the cobalt catalyst is known this calculated <d> can be corrected as follows:

$$\begin{matrix}{〈 d 〉}_{corrected} = {〈 d 〉}_{calc} \times ( DOR / 100 ) \\ = \frac{L \times 0.0674 \times ( DOR )}{{S}_{cat}}\end{matrix} \qquad ( 11 )$$

Where DOR=degree of reduction (% by weight). Thus equation (11) sets out a basis for determining the cobalt crystallite size.

### REFERENCES

^{‡} F. Delannay, “Characterisation of Heterogeneous Catalysts”, Marcel Dekker Inc., Chapter 7, 1984.
^{†} D. R. Lide, “Handbook of Chemistry and Physics”, CRC Press, 2007.
^{§} P. A. Webb and C. Orr, “Analytical Methods in Fine Particle Technology”, Micromeritic Instrument Corp., 1997.
S. Lovell, J. E. Shields, M. Thomas and M. Thommes, “Characterisation of Porous Solids and Powders: Surface Area, Pore Size and Density”, Kluwer Academic Publisher, 2004.

A particularly suitable hydrogen chemisorption method for determining surface area and cobalt crystallite size is as follows; approximately 0.2 to 0.5 g of sample material is firstly degassed and dried by heating to 140° C. at 10° C./min in flowing helium and maintaining at 140° C. for 60 minutes. The degassed and dried sample is then reduced by heating it from 140° C. to the desired reduction temperature at a rate of 3° C./min under a high flow of hydrogen (ca 200 ml/min) and then maintaining the hydrogen flow at that temperature for 6 hours. Following this reduction, the sample is heated under vacuum to 25° C. above the reduction temperature at a rate of 10° C./min and held under these conditions for 2 hours. The sample is then cooled to 150° C. and maintained for a further 30 minutes under vacuum. The chemisorption analysis is then carried out at 150° C. using pure hydrogen gas. An automatic analysis program is used to measure a full isotherm over the range 100 mm Hg up to 760 mm Hg pressure of hydrogen. The analysis is carried out twice; the first measures the “total” hydrogen uptake (i.e. includes chemisorbed hydrogen and physisorbed hydrogen) and immediately following the first analysis the sample is put under vacuum (<5 mm Hg) for 30 mins. The analysis is then repeated to measure the physisorbed uptake. A linear regression is then applied to the “total” uptake data with extrapolation back to zero pressure to calculate the volume of gas chemisorbed (V).

Cobalt surface areas were calculated in all cases using the following equation;

Co surface area=(6.023×10²³×V×SF×A)/22414

where
V=uptake of H₂ in ml/g
SF=Stoichiometry factor (assumed 2 for H₂ chemisorption on Co)
A=area occupied by one atom of cobalt (assumed 0.0662 nm²)

This equation is described in the Operators Manual for the Micromeritics ASAP 2010 Chemi System V 2.01, Appendix C, Part No. 201-42808-01, October 1996.

Catalysts in the reduced state can be difficult to handle as they can react spontaneously with oxygen in air, which can lead to undesirable self-heating and loss of activity. Consequently the reduced catalyst is preferably protected by encapsulation of the reduced catalyst particles with a suitable barrier coating. In the case of a Fischer-Tropsch catalyst, this may suitably be a hydrocarbon wax. The catalyst may them be provided in the form of a pellet, pastille or flake according to known methods. Alternatively the catalyst may be provided as a slurry in molten wax.

The catalyst may be prepared by the steps of:

(a) forming a catalyst precursor by;
(i) impregnating a transition alumina with a cobalt compound and precious metal promoter compound,
(ii) drying the impregnated alumina,
   (b) calcining the dried catalyst precursor, and
   (c) reducing the calcined precursor,
   wherein steps (i) and (ii) are repeated until the cobalt content of the catalyst is ≧25% by weight and the precious metal promoter content of the catalyst is in the range 0.05 to 0.25% by weight,
   the calcination is performed at a temperature in the range 250-650° C. and reduction is performed at a temperature in the range 450-650° C., such that cobalt crystallites in the catalyst have a average size, as determined by hydrogen chemisorption, of ≧15 nm.

In impregnation methods, a suitable soluble metal compound, for example the metal nitrate or acetate may be impregnated onto a support material from an aqueous or non-aqueous solution, e.g. ethanol, which may include other materials, and then dried to remove the solvent or solvents. One or more soluble metal compounds may be present in the solution. One or more impregnation steps may be performed to increase metal loading. Impregnation may be performed using any of the methods known to those skilled in the art of catalyst manufacture, but preferably is by way of a so-called ‘dry’ or ‘incipient-wetness’ impregnation as this minimises the quantity of solvent used and to be removed in drying. Incipient wetness impregnation comprises mixing the support material with only sufficient solution to fill the pores of the support. In the present invention, amounts up to 150% of incipient wetness volume are preferred. Impregnation methods for producing cobalt catalysts generally comprise combining a catalyst support with a solution of cobalt nitrate, e.g. cobalt (II) nitrate hexahydrate at a suitable concentration. Whereas a number of solvents may be used such as water, alcohols, ketones or mixtures of these, preferably the support has been impregnated using aqueous solutions of cobalt nitrate. With cobalt nitrate hexahydrate, it is possible to “self-solubilise” by warming the material to about 60° C. at which point the cobalt nitrate dissolves in its water of crystallisation. Preferably the impregnation and drying are repeated until the cobalt content of the resulting reduced catalyst is in the range 25-45% by weight.

The precious metal promoter is also included in the catalyst precursor by impregnation, using suitable soluble compounds such as the nitrate chloride, acetate, or mixtures of these. Preferably the precious metal promoter compound is a compound of Pt, Pd, Re, Ru, Ir or Au, and the impregnation is repeated until the precious metal content of the dried catalyst precursor is in the range in the range 0.05 to 0.25% by weight. In a preferred embodiment, the precious metal compound is a Ru compound, and the impregnation is repeated until the Ru content of the dried catalyst precursor is in the range in the range 0.05 to 0.15% by weight. Ruthenium nitrosyl nitrate is a particularly suitable Ru compound.

The cobalt compound and precious metal compound may be impregnated simultaneously or sequentially. Hence, the promoter may be included in the catalyst precursor before or after the cobalt, or at the same time by combining the cobalt and promoter compounds in the same impregnating solution.

The FT additives may be incorporated into the catalyst precursor by use of suitable compounds such as acids, metal salts, e.g. metal nitrates or metal acetates, or suitable metal-organic compounds, such as metal alkoxides or metal acetylacetonates. If desired, compounds of the additional additives may be added in suitable amounts to the cobalt and/or promoter solutions. Alternatively, the additive may be incorporated within the catalyst precursor in a separate step before or after drying or calcination.

The amount of cobalt and additive or promoter compound in solution, or the amount of transition alumina may be varied to achieve the desired metal loadings. Single or multiple impregnations may be performed to achieve the desired cobalt and additive or promoter levels in the catalyst precursor. In a preferred embodiment, the catalyst precursor is made by co-impregnating the transition alumina with an aqueous solution of ruthenium nitrosyl nitrate and cobalt (II) nitrate hexahyrate. In a particularly preferred method, cobalt (II) nitrate hexahydrate (Co content ca 20% wt) is heated to dissolve the nitrate in its own water of crystallisation and a 14-15% wt aqueous solution of Ru nitrosyl nitrate added to this to give the desired Co:Ru ratio.

The drying step may be performed at 20-120° C. in air or under an inert gas such as nitrogen, or in a vacuum oven. If desired, the catalyst precursor may be dried to remove solvent prior to calcination, or the calcination used to both dry and convert the catalyst precursor to the oxidic form. Prior to the high temperature calcination, the catalyst precursor may be pre-calcined at lower temperature, particularly after a first impregnation in advance of a second or further impregnation. Such low temperature pre-calcination is preferably performed by raising the temperature following the drying step to temperatures in the range 200-300° C. over periods of between 1 and 6 hours.

The catalyst precursor may then be calcined, i.e. heated in air or inert gas, to effect conversion of the cobalt and promoter compounds to their respective oxides. The calcination temperature is in the range 250 to 650° C., preferably 450 to 650° C., more preferably 450-550° C. The calcination time is preferably ≦24, more preferably ≦16, most preferably ≦8, especially 6 hours. Calcination is preferably performed by increasing the temperature over a period of 1-6 hours to a maximum temperature and holding there for a period up to about 6 hours.

To render the catalyst precursor catalytically active for Fischer-Tropsch reaction, at least a portion of the cobalt oxide may be reduced to the metal. The reducing step may be performed with a reducing gas selected from hydrogen, synthesis gas or a mixture of hydrogen and/or carbon monoxide with nitrogen or other inert gas. Preferred reducing gas streams that may be used include hydrogen- and/or carbon monoxide-containing gases. Reduction is preferably performed using hydrogen-containing gases at elevated temperature. Preferably the reducing gas stream comprises hydrogen at >25% vol, more preferably >50% vol, most preferably >75%, especially >90% vol hydrogen. In the present invention, the temperature of the reducing gas stream, and hence the catalyst precursor, during the reduction stage is in the range 450-650° C., preferably 475-650° C., more preferably 500 to 600° C. The reduction time is preferably ≦24, more preferably ≦16, most preferably ≦8, especially ≦6 hours, with a minimum reduction time of about 2 hours.

Preferably at least 75%, more preferably at least 85% of the cobalt is reduced, i.e. the degree of reduction (DOR) is preferably ≧75%, more preferably ≧85%, especially >90%, although this may be limited by cobalt aluminate formation. A temperature-programmed reduction (TPR) method for estimating DOR may be used as follows:

1. Steadily increase the sample temperature to the desired reduction temperature at 10° C./min, hold at that temperature for seven hours (TPR1).
2. Without cooling back to room temperature, increase the sample temperature to 1000° C. at 10° C./min and hold at 1000° C. for ten minutes. (TPR2). This gives complete reduction of all cobalt.
3. Integrate the hydrogen uptakes from TPRs 1 and 2. The ratio TPR1/(TPR1+TPR2) is the degree of reduction (expressed as %).

The reduction may be performed at ambient pressure or increased pressure, i.e. the pressure of the reducing gas may suitably be from 1-50, preferably 1-20, more preferably 1-10 bar abs.

The gas-hourly-space velocity (GHSV) for the reducing gas stream may be in the range 100-25000 hr⁻¹, preferably 1000-15000 hr⁻¹.

Before the reduction step, the dried or calcined catalyst precursor may, if desired, be formed into shaped units suitable for the process for which the catalyst is intended, using methods known to those skilled in the art. The shaped units may be agglomerates, spheres, pellets, cylinders, rings, or multi-holed pellets, which may be multi-lobed or fluted, e.g. of cloverleaf cross-section.

Moreover, following reduction, the process preferably further comprises a step of encapsulating the reduced catalyst in a hydrocarbon wax.

Hence, in a preferred embodiment, the catalyst may be prepared by the steps of:

(a) forming a catalyst precursor by;
(i) performing a first impregnation of a transition alumina with molten cobalt nitrate hexahydrate, optionally containing ruthenium nitrosyl nitrate,
(ii) drying the impregnated alumina,
(iii) pre-calcining the dried precursor by heating to 200-300° C.,
(iv) performing a second impregnation with molten cobalt nitrate hexahydrate containing ruthenium nitrosyl nitrate,
(v) drying the impregnated catalyst precursor,
   wherein sufficient Co and Ru are provided in steps (i) and (iv) such that the cobalt content of the catalyst is in the range 25-45% by weight and the ruthenium content of the catalyst is in the range 0.05 to 0.15% by weight,
   (b) calcining the dried catalyst precursor at a temperature in the range 250-650° C., preferably 450-650° C., and
   (c) reducing the calcined precursor at a temperature in the range 450-650° C., so that the cobalt crystallites in the catalyst have an average size ≧15 nm.

The catalysts may be used for the Fischer-Tropsch synthesis of hydrocarbons. The Fischer-Tropsch synthesis of hydrocarbons with cobalt catalysts is well established. The Fischer-Tropsch synthesis converts a mixture of carbon monoxide and hydrogen to hydrocarbons. The mixture of carbon monoxide and hydrogen is typically a synthesis gas having a hydrogen:carbon monoxide ratio in the range 1.6-3.0:1, preferably 1.7-2.5:1. The reaction may be performed in a continuous or batch process using one or more fixed bed reactors, stirred slurry-phase reactors, jet-loop reactors, bubble-column reactors, or fluidised bed reactors. The process may be operated at pressures in the range 0.1-10 Mpa and temperatures in the range 150-350° C. The gas-hourly-space velocity (GHSV) for continuous operation is in the range 100-25000 hr⁻¹. A preferred operating range is 1000-15000 hr⁻¹.

### EXAMPLES

The invention will now be further described by reference to the following Examples.

### Example 1

### Preparation of Catalyst

A commercially available high purity gamma alumina was used (Sasol SCFA-140). The Al₂O₃ content was 97.7% and loose bulk density was 0.58 g/ml. Physical property data is as follows;

| | Nitrogen Sorption data | | |
|---|---|---|---|
| | BET | | Median |
| | Surface | Pore | Pore |
| | Area | volume | Diameter |
| Sample | (m²/g) | (cm³/g) | (Å) |
| SCFA- | 142.9 | 0.47 | 132 |
| 140 | | | |

| | Particle size data | | |
|---|---|---|---|
| | D[v, 0.1] | D[v, 0.5] | D[v, 0.9] |
| Sample | (μm) | (μm) | (μm) |
| SCFA- | 4.5 | 20.4 | 44.6 |
| 140 | | | |

Catalysts were prepared by a two-stage impregnation process:

| | |
|---|---|
| 1st stage target | 18% wt Co (on final reduced catalyst) |
| Drying | 110° C./3 hrs |
| Pre-Calcination | 250° C./2 hrs |
| 2nd stage target | 30% wt Co + 0.1% wt Ru (on final reduced catalyst) |
| Drying | 110° C./3 hrs |
| Calcination | 250° C./2 hours or 500° C./4 hours. |

### 1^{st} Impregnation

300 g of alumina was placed into a z-blade mixer and 329.66 g molten cobalt (II) nitrate hexahydrate added while mixing. After mixing, the material had formed purple agglomerates. After the first impregnation the material was left to dry for 3 hours at 110° C. and then pre-calcined by heating from 110° C. to 250° C. at 2° C./min over 70 minutes and then held at this temperature for 2 hours. This material is referred to as P1.

### 2^{nd} Impregnation

300 g of the material P1 was placed into the z-blade mixer and a mixture of molten cobalt nitrate hexahydrate 204.98 g and 2.35 g of a 14.5% wt solution of ruthenium nitrosyl nitrate added while mixing. The resulting material was split into 2 batches. Both batches were dried at 110° C. for 3 hours but were calcined differently. The programmer was set as follows:

Ref: P2 heated from 110° C. to 250° C. over 70 minutes at 2° C./min, then held at 250° C. for 2 hours.
Ref: P3 heated from 110° C. to 500° C. over 78 minutes at 5° C./min, then held at 500° C. for 4 hours.

Comparative catalysts were also prepared according to the above method but without Ru (C1, C2 and C3 respectively).

Materials P1, P2, P3, C1, C2 and C3 were reduced in pure hydrogen according to the hydrogen chemisorption method described above in order to determine the Co surface area and crystallite size. The weight loss on reduction (WLOR) may be used to determine the Co content of the reduced catalyst. The results were as follows:

| | | | | Co wt % | |
|---|---|---|---|---|---|
| | Metal wt % | | | (reduced) calculated | |
| | content in calcined | | | based on WLOR at: | |
| | precursor by ICP OES | | | | 550° C. |
| | Al | | Co | 425° C. | according to |
| Sample | (% wt) | Ru (% wt) | (% wt) | comparative | invention |
| P1 | 38.2 | — | 16.2 | 18.1 | 18.1 |
| comparative | | | | | |
| P2 | 32.1 | 0.08 | 25.0 | 28.7 | 28.8 |
| P3 | 32.7 | 0.09 | 25.6 | 29.2 | 29.0 |
| C1 | 37.8 | — | 15.8 | 17.8 | — |
| comparative | | | | | |
| C2 | 31.6 | — | 24.6 | 28.8 | — |
| comparative | | | | | |
| C3 | 32.3 | — | 24.8 | 28.0 | 28.3 |
| comparative | | | | | |

Co loadings in the reduced catalysts may be deduced using the following equation:

$$L = \frac{Oxidic Co loading ( \% )}{( 100 - WLOR )} \times 100 \%$$

The Co surface areas (in m²/g catalyst) and weight loss on reduction (WLOR) for the various calcination and reduction conditions are shown in the table below:

Reduction Temp & WLOR (% w/w)

| | 425° C. | | | |
|---|---|---|---|---|
| Sample | comparative | WLOR % | 550° C. | WLOR % |
| P2 | 14.9 m²/g | 12.9 | 11.7 m²/g | 13.3 |
| P3 | 12.8 m²/g | 12.2 | 9.9 m²/g | 11.8 |

Using equation (11), the crystallite sizes may be then be determined.

$${〈 d 〉}_{corrected} = \frac{L \times 0.0674 \times ( DOR )}{{S}_{cat}} \qquad ( 11 )$$

Where

DOR=degree of reduction (%)
S_{cat}=Co Surface area per gram of catalyst (m²g_{cat}⁻¹)
L=reduced metal loading (%)

| | Reduction | L | DOR | S_{cat} | <d>_{corrected} |
|---|---|---|---|---|---|
| Sample | temp (° C.) | (%) | (%)^{c} | (m²g_{cat}⁻¹) | (nm) |
| P2 | 425 | 28.7 | 93 | 14.9 | 12.1 |
| | comparative | | | | |
| | 550 | 28.8 | 97 | 11.7 | 16.1 |
| P3 | 425 | 29.2 | 85 | 12.8 | 13.1 |
| | comparative | | | | |
| | 550 | 29.0 | 96 | 9.9 | 19.0 |

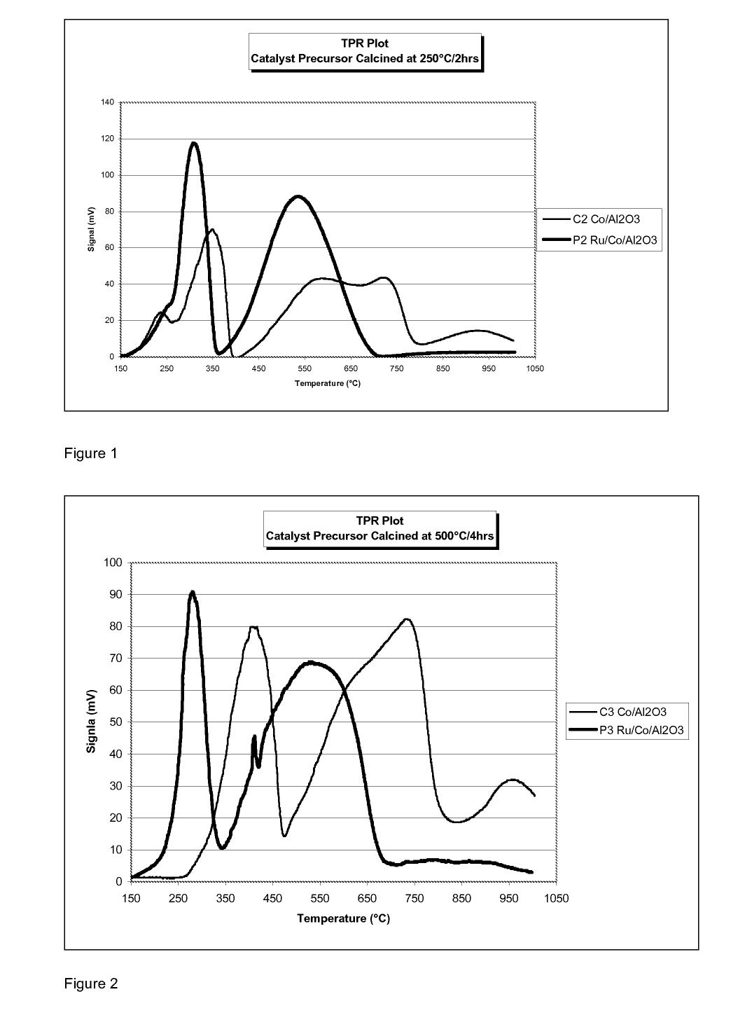

The TPR profiles of P2 and C2 are given in FIG. 1. The TPR profiles of P3 and C3 are given in FIG. 2. The effect of the Ru promoter in both cases is to reduce the temperature at which the CoO to Co metal reduction is effected. It can also be seen in both cases where the Ru is present that the cobalt aluminate reduction above 750° C. is absent.

### Calcined Catalyst Precursor Micromeritic Data

| | BET | Pore Vol | Average Pore | Particle Size | | |
|---|---|---|---|---|---|---|
| | Surface Area | [0.99ads] | Diameter | D[v, 0.1] | D[v, 0.5] | D[v, 0.9] |
| Sample Ref | (m²g⁻¹) | (cm³g⁻¹) | (Å) | (μm) | (μm) | (μm) |
| P1 | 112.1 | 0.31 | 109 | 8.8 | 23.1 | 46.7 |
| comparative | | | | | | |
| P2 | 89.4 | 0.23 | 104 | 9.2 | 23.4 | 46.7 |
| P3 | 82.4 | 0.24 | 117 | 9.0 | 23.2 | 47.0 |

### Example 2

### Catalyst Testing

a) Microreactor Testing

A fixed-bed microreactor consisting of six independent reactor tubes; each housed in its own furnace and supplied with gases (H₂, CO and Ar) from individual mass flow controllers. Catalyst temperature is measured and controlled using a thermocouple, placed at a fixed position, inside each packed bed. Reactants/products from each tube are fed into individual wax collection vessels, all housed inside a fan circulation oven—maintained at 130° C.—to trap out hydrocarbons >C15. Remaining gases from each tube (unconverted reactants and hydrocarbons <C15) feed into separate back-pressure regulators, all controlled using compressed air, to attain an upstream pressure of 20 bar. Downstream gases (at near atmospheric pressure) then feed into an 8-port sample valve where they can be selected for analysis by gas chromatography (using a Varian CP 3800 Gas Chromatograph). The sample valve is also supplied with a calibration gas feed and a nitrogen purge to flush the sample line. A data set for each reactor tube is collected every 3.5 hours for the duration of the experiment. Gases awaiting analysis flow through the sample valve and exit the oven, via cold-stage collection vessels—maintained at 6° C.—to condense any hydrocarbon-containing vapours and water, before flowing through individual rotameters to vent.

The glass reactor tubes used have an id=4 mm (od=8 mm and wall thickness=2 mm).

The testing procedure was as follows; 0.4 g P3 catalyst precursor in oxidic form plus 0.8 g powdered silicon carbide diluent were placed in a glass tube of id 4 mm and reduced at 425 or 550° C. in hydrogen (60 ml/min, SV=90001/kg Cat/hr). The temperature was steadily increased from 30° C. to either 425 or 550° C. at 3° C./min, dwelled for 7 hours, then cooled to 100° C. At 100° C., following reduction, the flow was switched from hydrogen to syngas:

64 ml/min (H₂:CO:Ar=40:20:4 ml/min), SV=9600 l/kgCat/hr, SV (excluding Ar)=9000 l/kgCat/hr. The pressure was increased from atmospheric to 20 bar and maintained at this pressure. The temperature was increased from 100° C. to 210° C. at 1° C./min; and held at 210° C. for duration of FT test. After 30 hours, the syngas flow was changed to 32 ml/min (H₂:CO:Ar=20:10:2 ml/min), SV=4800 l/kgCat/hr, SV (excluding Ar)=4500 l/kgCat/hr. The test then continued for a further 65 hours.

Data taken 20 hrs into experiment (at initial flow rate)

| | Reduction | Total Flow | SV |
|---|---|---|---|
| Catalyst | Temp (° C.) | (ml/min) | (l/kgCat/hr) |
| P3 | 425 | 64 | 9600 |
| P3 | 550 | 64 | 9600 |

| Catalyst | % Conversion | % CH₄ | % CO₂ | % C2-C4 | % C5+ |
|---|---|---|---|---|---|
| P3 | 32.51 | 7.73 | 0.8 | 8.14 | 83.33 |
| P3 | 26.88 | 8.88 | 0.77 | 9.04 | 81.31 |

Data taken 90 hrs into experiment (at changed flow rate)

| | Reduction | Total Flow | SV |
|---|---|---|---|
| Catalyst | Temp (° C.) | (ml/min) | (l/kgCat/hr) |
| P3 | 425 | 32 | 4800 |
| P3 | 550 | 32 | 4800 |

| Catalyst | % Conversion | % CH₄ | % CO₂ | % C2-C4 | % C5+ |
|---|---|---|---|---|---|
| P3 | 49.89 | 6.73 | 0.28 | 8.87 | 84.12 |
| P3 | 47.03 | 7.23 | 0.22 | 8.43 | 84.12 |

The GC analysis was used to determine the amount of C5+ hydrocarbons formed, which is an accepted measure for selectivity to desired hydrocarbons. The results demonstrate excellent selectivity, particularly considering;
(a) the particle size of the catalyst is below that which published work suggests is optimum for selectivity (see Iglesia et al, Advances in Catalysis 1993, 39, pages 221-302), and
(b) the average cobalt crystallite size is considerably larger than previous work suggests is optimum for selectivity (see Borg et al, J. Catal, 259, 2008, pages 161-164).

At these space velocities, the activity of the catalyst is suitable for industrial use. The activity has not been compromised by using the high calcination and reduction temperatures.

### Example 3

### Catalyst Preparation

A further catalyst was prepared according to the method for catalyst P3 on a larger scale, except that Ru was added in both impregnation steps.

### 1^{st} Impregnation.

7700 g of molten cobalt nitrate hexahydrate and 34.63 g of a 15.07% aqueous solution of ruthenium nitrosyl nitrate were combined and sprayed onto 7000 g of the gamma alumina. The material was dried at 110° C. for 6 hours, and then the dried catalyst precursor was pre-calcined at 250° C. for 2 hours using the same method as for P3.

### 2^{nd} Impregnation

9000 g of the dried and pre-calcined material were then impregnated with a mixture of 7200 g of molten cobalt nitrate hexahydrate and 32.05 g of 15.07% aqueous ruthenium nitrosyl nitrate solution. The resulting catalyst precursor was dried at 110° C. for 6 hours and then the material calcined at 500° C. for 4 hours according to the method for P3. The resulting catalyst precursor reference was P4.

Samples were exposed to different reduction temperatures in the range 450-550° C., in hydrogen using the hydrogen chemisorption technique. The results were as follows;

| Reduction | L | DOR | S_{cat} | <d>_{corrected} |
|---|---|---|---|---|
| temp (° C.) | (%) | (%) | (m²g_{cat}⁻¹) | (nm) |
| 450 | 31.2 | 87.7 | 11.9 | 15.5 |
| 475 | 31.2 | 94.1 | 11.2 | 17.6 |
| 500 | 31.5 | 96.0 | 10.6 | 19.2 |
| 525 | 31.5 | 96.6 | 9.3 | 22.1 |
| 550 | 31.2 | 97.1 | 9.2 | 22.2 |

The results show the effect of increasing the reduction temperature on Co crystallite size.

Although the invention is illustrated and described herein with reference to specific embodiments, the invention is not intended to be limited to the details shown. Rather, various modifications may be made in the details within the scope and range of equivalents of the claims and without departing from the invention.

1. A catalyst suitable for the Fischer-Tropsch synthesis of hydrocarbons, comprising cobalt crystallites containing a precious metal promoter, dispersed over a surface of a porous transition alumina powder, wherein a cobalt content of the catalyst is ≧25% by weight, a precious metal promoter content of the catalyst is in the range 0.05 to 0.25% by weight, and the cobalt crystallites have an average size, as determined by hydrogen chemisorption, of ≧15 nm.

2. A catalyst according to claim 1 wherein the cobalt content of the catalyst is in the range 25 to 45% by weight.

3. A catalyst according to claim 1 wherein the cobalt crystallites have an average size of ≧17.5 nm.

4. A catalyst according to claim 1 wherein the promoter is selected from one or more of the group consisting of Pt, Pd, Re, Ru, Ir and Au.

5. A catalyst according to claim 1 wherein the promoter comprises Ru, which is present in an amount in the range 0.05 to 0.15% by weight.

6. A catalyst according to claim 1 wherein the transition alumina comprises an alumina selected from the group consisting of a gamma alumina and a delta alumina.

7. A catalyst according to claim 6 wherein the alumina has an average particle size ≦25 μm.

8. A catalyst according to claim 6 wherein the BET surface area of the alumina is in the range 120 to 160 m²/g.

9. A catalyst according to claim 6 wherein the alumina has a pore volume in the range 0.35 to 0.65 cm³/g.

10. A catalyst according to claim 6 wherein the alumina has an average pore diameter of ≧10 nm.

11. A catalyst according to claim 1 further comprising one or more additive compound of metals selected from the group consisting of molybdenum, iron, manganese, titanium, zirconium, lanthanum, cerium, chromium, magnesium and zinc.

12. A catalyst according to claim 1 encapsulated in a hydrocarbon wax.

13. A process for preparing a catalyst comprising the steps of:
(a) forming a catalyst precursor by;
(i) impregnating a transition alumina with a cobalt compound and a precious metal promoter compound, and
(ii) drying the impregnated alumina to produce a dried catalyst precursor,
(b) calcining the dried catalyst precursor at a temperature in the range 250 to 650° C. to produce a calcined precursor, and
(c) reducing the calcined precursor at a temperature in the range 450 to 650° C.,
wherein steps (i) and (ii) are performed until a cobalt content of the catalyst is ≧25% by weight and a precious metal promoter content of the catalyst is in the range 0.05 to 0.25% by weight, such that cobalt crystallites in the catalyst have an average size, as determined by hydrogen chemisorption, of ≧15 nm.

14. A process according to claim 13 wherein impregnation and drying are repeated until the cobalt content of the catalyst is in the range 25 to 45% by weight.

15. A process according to claim 13 wherein the cobalt crystallites have an average size of ≧17.5 nm.

16. A process according to claim 13 wherein the cobalt compound and the precious metal compound are impregnated simultaneously.

17. A process according to claim 13 wherein the cobalt compound and the precious metal compound are impregnated sequentially.

18. A process according to claim 13 wherein the precious metal compound is selected from a compound selected from the group consisting of compounds of Pt, Pd, Re, Ru, Ir and Au.

19. A process according to claim 13 wherein the precious metal compound is a Ru compound, and the impregnation is repeated until a Ru content of the catalyst is in the range 0.05 to 0.15% by weight.

20. A process according to claim 13 wherein the transition alumina comprises an alumina selected from the group consisting of a gamma alumina and a delta alumina.

21. A process according to claim 20 wherein the alumina has an average particle size ≦25 μm.

22. A process according to claim 20 wherein the BET surface area of the alumina is in the range 120 to 160 m²/g.

23. A process according to claim 20 wherein the alumina has a pore volume in the range 0.35 to 0.65 cm³/g.

24. A process according to claim 20 wherein the alumina has an average pore diameter of ≧10 nm.

25. A process according to claim 13 wherein one or more additive compounds of metals selected from the group consisting of molybdenum, iron, manganese, titanium, zirconium, lanthanum, cerium, chromium, magnesium and zinc is also impregnated into the catalyst support either simultaneously or sequentially with the cobalt compound.

26. A process according to claim 13 where two or more impregnation stages are performed, further comprising a pre-calcination stage performed on the dried precursor between each of the impregnations.

27. A process according to claim 13 wherein the calcination is performed in air at a temperature in the range 450 to 650° C.

28. A process according to claim 13 wherein the reduction is performed at a temperature in the range 500 to 600° C.

29. A process according to claim 13 wherein the reducing step is performed with a reducing gas selected from the group consisting of hydrogen, synthesis gas, and a mixture of hydrogen or carbon monoxide with nitrogen or other inert gas.

30. A process according to claim 29 wherein the reducing gas mixture comprises >90% vol hydrogen.

31. A process according to claim 13 further comprising a step of encapsulating the reduced catalyst in a hydrocarbon wax.

32. A process for the Fischer-Tropsch synthesis of hydrocarbons comprising the step of passing a gas mixture comprising hydrogen and carbon monoxide over a catalyst in a Fischer-Tropsch reactor, the catalyst comprising cobalt crystallites containing a precious metal promoter, dispersed over the surface of a porous transition alumina powder, wherein a cobalt content of the catalyst is ≧25% by weight, a precious metal promoter content of the catalyst is in the range 0.05 to 0.25% by weight, and the cobalt crystallites have an average size, as determined by hydrogen chemisorption, of ≧15 nm.

33. A process according to claim 32 wherein the mixture of carbon monoxide and hydrogen is a synthesis gas having a hydrogen:carbon monoxide ratio in the range 1.6 to 3.0:1.

34. A process according to claim 32 wherein a reaction is operated at pressures in the range 0.1 to 10 Mpa and temperatures in the range 150 to 350° C.

35. A process according to claim 32 wherein the Fischer Tropsch reactor is a fixed bed reactor or a slurry phase reactor.

36. A catalyst according to claim 1 wherein the cobalt crystallites have an average size of ≧20 nm.

37. A process according to claim 13 wherein the cobalt crystallites have a average size of ≧20 nm.
